(19) United States
(12) Patent Application Publication
(10) Pub. No.: US 2013/0178663 A1
(43) Pub. Date: Jul. 11, 2013

(54) COBALT-CONTAINING HYDROGENATION CATALYSTS AND PROCESSES FOR MAKING SAME

(71) Applicants: Zhenhua Zhou, Houston, TX (US); Dheeraj Kumar, Pearland, TX (US); Xiaoyan Tu, Blacksburg, VA (US); Heiko Weiner, Pasadena, TX (US); Radmila Wollrab, Pasadena, TX (US)

(72) Inventors: Zhenhua Zhou, Houston, TX (US); Dheeraj Kumar, Pearland, TX (US); Xiaoyan Tu, Blacksburg, VA (US); Heiko Weiner, Pasadena, TX (US); Radmila Wollrab, Pasadena, TX (US)

(73) Assignee: CELANESE INTERNATIONAL CORPORATION, Irving, TX (US)

(21) Appl. No.: 13/595,358

(22) Filed: Aug. 27, 2012

Related U.S. Application Data
(60) Provisional application No. 61/583,922, filed on Jan. 6, 2012.

(51) Int. Cl. B01J 23/89; C07C 27/04; B01J 23/888

(52) U.S. Cl. 568/885; 502/310; 502/242; 502/254; 502/313

(57) ABSTRACT

The present invention relates to catalysts, to processes for making catalysts and to chemical processes employing such catalysts. The catalysts are preferably used for converting acetic acid to ethanol. The catalyst comprises cobalt, precious metal and one or more active metals on a modified support.

### CROSS REFERENCE TO RELATED APPLICATION

This application claims priority to U.S. Provisional App. No. 61/583,922, filed on Jan. 6, 2012, the entirety of which is incorporated herein by reference.

### FIELD OF THE INVENTION

The present invention relates to catalysts, to processes for making catalysts, and to processes for producing ethanol from a feed stream comprising a carboxylic acid and/or esters thereof in the presence of the inventive catalysts. In one embodiment the catalyst comprises cobalt on a modified support.

### BACKGROUND OF THE INVENTION

Ethanol for industrial use is conventionally produced from petrochemical feed stocks, such as oil, natural gas, or coal, from feed stock intermediates, such as syngas, or from starchy materials or cellulosic materials, such as corn or sugar cane. Conventional methods for producing ethanol from petrochemical feed stocks, as well as from cellulosic materials, include the acid-catalyzed hydration of ethylene, methanol homologation, direct alcohol synthesis, and Fischer-Tropsch synthesis. Instability in petrochemical feed stock prices contributes to fluctuations in the cost of conventionally produced ethanol, making the need for alternative sources of ethanol production all the greater when feed stock prices rise. Starchy materials, as well as cellulosic material, are converted to ethanol by fermentation. However, fermentation is typically used for consumer production of ethanol, which is suitable for fuels or human consumption. In addition, fermentation of starchy or cellulosic materials competes with food sources and places restraints on the amount of ethanol that can be produced for industrial use.

Ethanol production via the reduction of alkanoic acids and/or other carbonyl group-containing compounds has been widely studied, and a variety of combinations of catalysts, supports, and operating conditions have been mentioned in the literature. The reduction of various carboxylic acids over metal oxides has been proposed by EP0175558 and U.S. Pat. No. 4,398,039. A summary some of the developmental efforts for hydrogenation catalysts for conversion of various carboxylic acids is provided in Yokoyama, et al., “Carboxylic acids and derivatives” in: Fine Chemicals Through Heterogeneous Catalysis, 2001, 370-379.

U.S. Pat. No. 8,080,694 describes a process for hydrogenating alkanoic acids comprising passing a gaseous stream comprising hydrogen and an alkanoic acid in the vapor phase over a hydrogenation catalyst comprising: a platinum group metal selected from the group consisting of platinum, palladium, rhenium and mixtures thereof on a silicaceous support; and a metallic promoter selected the group consisting of tin, rhenium and mixtures thereof, the silicaceous support being promoted with a redox promoter selected from the group consisting of: WO₃; MoO₃; Fe₂O₃ and Cr₂O₃.

U.S. Pat. No. 7,608,744 describes a process for the selective production of ethanol by vapor phase reaction of acetic acid at a temperature of about 250° C. over a hydrogenating catalyst composition either cobalt and palladium supported on graphite or cobalt and platinum supported on silica selectively produces ethanol.

U.S. Pat. No. 6,495,730 describes a process for hydrogenating carboxylic acid using a catalyst comprising activated carbon to support active metal species comprising ruthenium and tin. U.S. Pat. No. 6,204,417 describes another process for preparing aliphatic alcohols by hydrogenating aliphatic carboxylic acids or anhydrides or esters thereof or lactones in the presence of a catalyst comprising Pt and Re. U.S. Pat. No. 5,149,680 describes a process for the catalytic hydrogenation of carboxylic acids and their anhydrides to alcohols and/or esters in the presence of a catalyst containing a Group VIII metal, such as palladium, a metal capable of alloying with the Group VIII metal, and at least one of the metals rhenium, tungsten or molybdenum. U.S. Pat. No. 4,777,303 describes a process for the productions of alcohols by the hydrogenation of carboxylic acids in the presence of a catalyst that comprises a first component which is either molybdenum or tungsten and a second component which is a noble metal of Group VIII on a high surface area graphitized carbon. U.S. Pat. No. 4,804,791 describes another process for the production of alcohols by the hydrogenation of carboxylic acids in the presence of a catalyst comprising a noble metal of Group VIII and rhenium. U.S. Pat. No. 4,517,391 describes preparing ethanol by hydrogenating acetic acid under superatmospheric pressure and at elevated temperatures by a process wherein a predominantly cobalt-containing catalyst.

Existing processes suffer from a variety of issues impeding commercial viability including: (i) catalysts without requisite selectivity to ethanol; (ii) catalysts which are possibly prohibitively expensive and/or nonselective for the formation of ethanol and that produce undesirable by-products; (iii) required operating temperatures and pressures which are excessive; (iv) insufficient catalyst life; and/or (v) required activity for both ethyl acetate and acetic acid.

### SUMMARY OF THE INVENTION

The invention is generally directed to catalysts, to processes for forming catalysts and to processes for employing the catalysts in a hydrogenation process. In one embodiment, the invention is to a catalyst, comprising first, second and third metals on a modified support, wherein the first metal is a precious metal, and provided that at least one of the second or third metals is cobalt, and wherein the modified support comprises a support modifier metal selected from the group consisting of tungsten, molybdenum, vanadium, niobium, and tantalum.

In a first embodiment, the invention is directed to a catalyst comprising cobalt, a precious metal and at least one active metal on a modified support, wherein the precious metal is selected from the group consisting of rhodium, rhenium, ruthenium, platinum, palladium, osmium, iridium and gold; wherein the at least one active metal is selected from the group consisting of copper, iron, nickel, titanium, zinc, chromium, tin, lanthanum, cerium, and manganese; and wherein the modified support comprises (i) support material; (ii) a support modifier comprising a metal selected from the group consisting of tungsten, molybdenum, vanadium, niobium, and tantalum. In one embodiment, the support modifier is an oxide of tungsten, molybdenum, or a mixture thereof. In another embodiment, the support modified is an oxide of vanadium, niobium, tantalum, or mixtures thereof. In one embodiment the modified support is substantially free of cobalt and/or the active metal. It is understood that even though the modified support does not contain cobalt and/or the active metal, these metals, along with the precious, are on the modified support.

For example, the catalyst may comprise the precious metal in an amount from 0.1 to 5 wt. %, cobalt in an amount from 0.5 to 20 wt. %, e.g., preferably from 4.1 to 20 wt. %, and tin in an amount from 0.5 to 20 wt. %, e.g., preferably from 0.5 to 3.5 wt. %. In one aspect, the precious metal is palladium, and the one or more active metals comprise cobalt and tin, and in another aspect the precious metal is platinum, and the one or more active metals comprise cobalt and tin.

The support itself preferably is a silicaceous support, e.g., silica, or a carbon support, e.g., carbon black or activated carbon, although any of a variety of other supports may be used. In various embodiments, for example, the support may be selected from silica, alumina, titania, silica/alumina, calcium metasilicate, pyrogenic silica, silica gel, high purity silica, zirconia, carbon, zeolites and mixtures thereof. The support modifier may comprise tungsten in a variety of forms, such as in the form of tungsten oxide.

In a second embodiment, the invention is directed to a catalyst, comprising: a modified support comprising a silicaceous support material and a support modifier comprising a support modifier metal selected from the group consisting of tungsten, molybdenum, niobium, vanadium and tantalum, and a first metal, a second metal and a third metal on the modified support, wherein the first metal is a precious metal, and wherein the first metal is present in an amount from 0.1 to 5 wt. %, the second metal is present in an amount from 0.5 to 20 wt. % and the third metal is present in an amount from 0.5 to 20 wt. %, based on the total weight of the catalyst, provided that at least one of the second or third metals is cobalt. The second or third metals are preferably different and may be active metals selected from the group consisting of cobalt, copper, iron, nickel, titanium, zinc, chromium, tin, lanthanum, cerium, and manganese.

In another embodiment, the invention is to a process for forming a catalyst, the process comprising the steps of: (a) impregnating a support with a support modifier precursor to form a first impregnated support, wherein the support modifier precursor comprises a support modifier metal selected from the group consisting of tungsten, molybdenum, niobium, vanadium and tantalum; (b) heating the first impregnated support to a first temperature to form a modified support; (c) impregnating the modified support with a second mixed precursor to form a second impregnated support, wherein the second mixed precursor comprises a first metal precursor, a second metal precursor, and a third metal precursor, provided that one of the second metal precursors or third metal precursors comprises cobalt; and (d) heating the second impregnated support to a second temperature to form the catalyst. The second temperature preferably is less than the first temperature, e.g., at least 50° C. less than the first temperature, or at least 100° C. less than the first temperature.

In another embodiment, the invention is to a process for producing ethanol, comprising contacting a feed stream comprising acetic acid and/or ethyl acetate, and hydrogen in a reactor at an elevated temperature in the presence of any of the above-described catalysts, under conditions effective to form ethanol. The feed stream optionally further comprises ethyl acetate in an amount greater than 5 wt. %. Acetic acid conversion optionally is greater than 20%, e.g., greater than 50%, greater than 80% or greater than 90%, and ethyl acetate conversion optionally is greater than 5%, greater than 10% or greater than 15%. Acetic acid selectivity to ethanol optionally is greater than 80% or greater than 90%. In a preferred aspect, the process forms a crude product comprising the ethanol and ethyl acetate, and the crude product has an ethyl acetate steady state concentration from 0.1 to 40 wt. %, e.g., from 0.1 to 20 wt. % or from 0.1 to 10 wt. %. The hydrogenation optionally is performed in a vapor phase at a temperature of from 125° C. to 350° C., a pressure of 10 kPa to 3000 kPa, and a hydrogen to acetic acid mole ratio of greater than 4:1. The acetic acid optionally is derived from a carbonaceous material selected from the group consisting of oil, coal, natural gas and biomass.

In a third embodiment, the invention is directed to a hydrogenation catalyst comprising cobalt, a precious metal and at least one active metal on a modified support comprising tungsten oxide, and having, after calcination, an x-ray diffraction pattern substantially as shown Table 4. Preferably, the precious metal is selected from the group consisting of rhodium, rhenium, ruthenium, platinum, palladium, osmium, iridium and gold and the at least one active metal is selected from the group consisting of copper, iron, nickel, titanium, zinc, chromium, tin, lanthanum, cerium, and manganese.

In a fourth embodiment, the invention is directed to a catalyst comprising cobalt, a precious metal and at least one active metal on a modified support comprising tungsten oxide, and having, after calcination, an x-ray diffraction pattern in which above 2θ=10°, there is a local maximum having a characteristic full width at a half maximum at each of: a 2θ value in the range from 23.54 to 24.60°; a 2θ value in the range from 27.81 to 28.13°; a 2θ value in the range from 33.52 to 34.56°; a 2θ value in the range from 41.62 to 42.42°; a 2θ value in the range from 54.70 to 55.66°; a 2θ value in the range from 60.18 to 61.32°. Preferably, the precious metal is selected from the group consisting of rhodium, rhenium, ruthenium, platinum, palladium, osmium, iridium and gold and the at least one active metal is selected from the group consisting of copper, iron, nickel, titanium, zinc, chromium, tin, lanthanum, cerium, and manganese.

### BRIEF DESCRIPTION OF THE DRAWINGS

The present invention will be better understood in view of the appended non-limiting figures, in which:

FIG. 1 provides a non-limiting flow diagram for a process for forming a catalyst according to one embodiment of the present invention.

FIG. 2 is a graph showing performance of the Catalyst of Example 5 under standard running conditions.

FIG. 3 is a graph showing performance of a comparative catalyst under standard running conditions.

FIG. 4 is an XRD plot for the catalyst of Example 5-7.

### DETAILED DESCRIPTION OF THE INVENTION

### Catalyst Composition

The present invention is directed to catalyst compositions that preferably are suitable as hydrogenation catalysts, to processes for forming such catalysts, and to chemical processes employing such catalysts. The catalysts preferably comprise one or more active metals, and in particular cobalt, on a support, preferably a modified support, and may be suitable in catalyzing the hydrogenation of a carboxylic acid, e.g., acetic acid, and/or esters thereof, e.g., ethyl acetate, to the corresponding alcohol, e.g., ethanol.

In one embodiment, the inventive catalyst comprises cobalt, a precious metal and at least one active metal on a modified support. Preferably the support is a modified support comprising a support material and a support modifier, wherein the support modifier comprises a metal selected from tungsten, molybdenum, vanadium, niobium and tantalum. In one aspect, the modified support is substantially free of cobalt and/or active metals. It is understood that even though the modified support does not contain cobalt and/or the active metal, these metals, along with the precious metal, may be loaded on the modified support after the support modifier is calcined on the support material.

It has now been discovered that such catalysts are particularly effective as multifunctional hydrogenation catalysts capable of converting both carboxylic acids, such as acetic acid, and esters thereof, e.g., ethyl acetate, to their corresponding alcohol(s), e.g., ethanol, under hydrogenation conditions. Thus, in another embodiment, the inventive catalyst comprises a precious metal and an active metal on a modified support, wherein the catalyst is effective for providing an acetic acid conversion greater than 20%, greater than 75% or greater than 90%, and an ethyl acetate conversion greater than 0%, greater than 10% or greater than 20%.

### Precious and Active Metals

In addition to cobalt, the catalysts of the invention preferably include at least one precious metal impregnated on the catalyst support. The precious metal may be selected, for example, from rhodium, rhenium, ruthenium, platinum, palladium, osmium, iridium and gold. Preferred precious metals for the catalysts of the invention include palladium, platinum, and rhodium. The precious metal preferably is catalytically active in the hydrogenation of a carboxylic acid and/or its ester to the corresponding alcohol(s). The precious metal may be in elemental form or in molecular form, e.g., an oxide of the precious metal. It is preferred that the catalyst comprises such precious metals in an amount less than 5 wt. %, e.g., less than 3 wt. %, less than 2 wt. %, less than 1 wt. % or less than 0.5 wt. %. In terms of ranges, the catalyst may comprise the precious metal in an amount from 0.05 to 10 wt. %, e.g. from 0.1 to 5 wt. %, or from 0.1 to 3 wt. %, based on the total weight of the catalyst. In some embodiments, the metal loading of the precious metal may be less than the metal loadings of cobalt or the one or more active metals.

The catalyst also includes at least one active metals impregnated on the support. When multiple active metals are used, at least one of the active metals is cobalt. As used herein, active metals refer to catalytically active metals that improve the conversion, selectivity and/or productivity of the catalyst and may include precious or non-precious active metals. Thus, a catalyst comprising a precious metal and an active metal may include: (i) one (or more) precious metals and one (or more) non-precious active metals, or (ii) may comprise two (or more) precious metals. Thus, precious metals are included herein as exemplary active metals. Further, it should be understood that use of the term “active metal” to refer to some metals in the catalysts of the invention is not meant to suggest that the precious metal that is also included in the inventive catalysts is not catalytically active.

In one embodiment, the one or more active metals included in the catalyst are selected from the group consisting of copper, iron, nickel, titanium, zinc, chromium, tin, lanthanum, cerium, and manganese, or from any of the aforementioned precious metals. The active metals may also include cobalt when multiple active metals are used. Preferably, however, the one or more active metals do not include any precious metals. More preferably, the one or more active metals are selected from the group consisting of copper, iron, nickel, zinc, chromium, and tin. The one or more active metals may be in elemental form or in molecular form, e.g., an oxide of the active metal, or a combination thereof.

The total weight of all the catalytic metals, including precious metals, active metals, and cobalt, present in the catalyst preferably is from 0.1 to 25 wt. %, e.g., from 0.5 to 15 wt. %, or from 1.0 to 10 wt. %. In one embodiment, the catalyst may comprise from cobalt in an amount from 0.5 to 20 wt. %, e.g., preferably from 4.1 to 20 wt. %, and tin in an amount from 0.5 to 20 wt. %, e.g., preferably from 0.5 to 3.5 wt. %. The active metals for purposes of the present invention may be disposed on the modified support and are not a part of the modified support. For purposes of the present specification, unless otherwise indicated, weight percent is based on the total weight the catalyst including metal and support.

In some embodiments, the catalyst contains at least two active metals in addition to the precious metal, provided that one of the active metals is cobalt. The at least two active metals may be selected from any of the active metals identified above, so long as they are not the same as the precious metal or each other. Additional active metals may also be used in some embodiments. Thus, in some embodiments, there may be multiple active metals on the support in addition to the precious metal.

Exemplary tertiary combinations may include cobalt/rhodium/copper, cobalt/rhodium/iron, cobalt/rhodium/nickel, cobalt/rhodium/chromium, cobalt/rhodium/tin, cobalt/rhenium/copper, cobalt/rhenium/nickel, cobalt/rhenium/tin, cobalt/ruthenium/copper, cobalt/ruthenium/nickel, cobalt/ruthenium/tin, cobalt/platinum/copper, cobalt/platinum/iron, cobalt/platinum/nickel, cobalt/platinum/chromium, cobalt/platinum/tin, cobalt/platinum/zinc, cobalt/platinum/titanium, cobalt/palladium/copper, cobalt/palladium/iron, cobalt/palladium/nickel, cobalt/palladium/chromium, cobalt/palladium/tin, cobalt/osmium/copper, cobalt/osmium/nickel, cobalt/osmium/tin, cobalt/iridium/copper, cobalt/iridium/nickel, cobalt/iridium/tin, cobalt/gold/copper, cobalt/gold/nickel, and cobalt/gold/tin.

In one preferred embodiment, the tertiary combination comprises cobalt and tin. In some embodiments, the catalyst may comprise more than three metals on the support.

When the catalyst comprises a precious metal, cobalt, and an active metal on a support, the active metal is present in an amount from 0.1 to 20 wt. %, e.g., from 0.1 to 10 wt. %, or from 0.1 to 7.5 wt. %. Cobalt may be present in an amount from 4.1 to 20 wt. %, e.g., from 4.1 to 10 wt. % or from 4.1 to 7.5 wt. %. When the catalyst comprises two or more active metals in addition to the precious metal, the first active metal may be present in the catalyst in an amount from 0.05 to 20 wt. %, e.g. from 0.1 to 10 wt. %, or from 0.5 to 7.5 wt. %. If the catalyst further comprises a second or third active metal may be present in an amount from 0.05 to 20 wt. %, e.g., from 0.1 to 10 wt. %, or from 0.5 to 7.5 wt. %. The active metals may be alloyed with one another or may comprise a non-alloyed metal solution, a metal mixture or be present as one or more metal oxides.

The preferred metal ratios may vary somewhat depending on the active metals used in the catalyst. In some embodiments, the mole ratio of the precious metal to the one or more active metals is from 10:1 to 1:10, e.g., from 4:1 to 1:4, from 2:1 to 1:2 or from 1.5:1 to 1:1.5. In another embodiment, the precious metal may be present in an amount from 0.1 to 5 wt. %, cobalt in an amount from 0.5 to 20 wt. % and the second active metal in an amount from 0.5 to 20 wt. %, based on the total weight of the catalyst. In another embodiment, the precious metal is present in an amount from 0.1 to 5 wt. %, cobalt in an amount from 0.5 to 7.5 wt. % and the active metal in an amount from 0.5 to 7.5 wt. %.

In one embodiment, the first and second active metals are present as cobalt and tin, and, when added to the catalyst together and calcined together, are present at a cobalt to tin molar ratio from 6:1 to 1:6 or from 3:1 to 1:3. The cobalt and tin may be present in substantially equimolar amounts, when added to the catalyst together and calcination together. In another embodiment, when cobalt is added to the support material initially and calcined as part of the modified support and tin is subsequently added to the modified support, it is preferred to have a cobalt to tin molar that is greater than 4:1, e.g., greater than 6:1 or greater than 11:1. Without being bound by theory the excess cobalt, based on molar amount relative to tin, may improve the multifunctionality of the catalyst.

### Support Materials

The catalysts of the present invention comprise a suitable support material, preferably a modified support material. In one embodiment, the support material may be an inorganic oxide. In one embodiment, the support material may be selected from the group consisting of silica, alumina, titania, silica/alumina, pyrogenic silica, high purity silica, zirconia, carbon (e.g., carbon black or activated carbon), zeolites and mixtures thereof. Preferably, the support material comprises a silicaceous support material such as silica, pyrogenic silica, or high purity silica. In one embodiment the silicaceous support material is substantially free of alkaline earth metals, such as magnesium and calcium. In preferred embodiments, the support material is present in an amount from 25 wt. % to 99 wt. %, e.g., from 30 wt. % to 98 wt. % or from 35 wt. % to 95 wt. %, based on the total weight of the catalyst.

In preferred embodiments, the support material comprises a silicaceous support material, e.g., silica, having a surface area of at least 50 m²/g, e.g., at least 100 m²/g, or at least 150 m²/g. In terms of ranges, the silicaceous support material preferably has a surface area from 50 to 600 m²/g, e.g., from 100 to 500 m²/g or from 100 to 300 m²/g. High surface area silica, as used throughout the application, refers to silica having a surface area of at least 250 m²/g. For purposes of the present specification, surface area refers to BET nitrogen surface area, meaning the surface area as determined by ASTM D6556-04, the entirety of which is incorporated herein by reference.

The preferred silicaceous support material also preferably has an average pore diameter from 5 to 100 nm, e.g., from 5 to 30 nm, from 5 to 25 nm or from 5 to 10 nm, as determined by mercury intrusion porosimetry, and an average pore volume from 0.5 to 2.0 cm³/g, e.g., from 0.7 to 1.5 cm³/g or from 0.8 to 1.3 cm³/g, as determined by mercury intrusion porosimetry.

The morphology of the support material, and hence of the resulting catalyst composition, may vary widely. In some exemplary embodiments, the morphology of the support material and/or of the catalyst composition may be pellets, extrudates, spheres, spray dried microspheres, rings, pentarings, trilobes, quadrilobes, multi-lobal shapes, or flakes although cylindrical pellets are preferred. Preferably, the silicaceous support material has a morphology that allows for a packing density from 0.1 to 1.0 g/cm³, e.g., from 0.2 to 0.9 g/cm³ or from 0.3 to 0.8 g/cm³. In terms of size, the silica support material preferably has an average particle size, meaning the average diameter for spherical particles or average longest dimension for non-spherical particles, from 0.01 to 1.0 cm, e.g., from 0.1 to 0.7 cm or from 0.2 to 0.5 cm. Since the precious metal and the one or more active metals that are disposed on the support are generally in the form of very small metal (or metal oxide) particles or crystallites relative to the size of the support, these metals should not substantially impact the size of the overall catalyst particles. Thus, the above particle sizes generally apply to both the size of the support as well as to the final catalyst particles, although the catalyst particles are preferably processed to form much larger catalyst particles, e.g., extruded to form catalyst pellets.

### Support Modifiers

The support material preferably comprises a support modifier. A support modifier may adjust the acidity of the support material. In another embodiment, the support modifier may be a basic modifier that has a low volatility or no volatility. In one embodiment, the support modifiers are present in an amount from 0.1 wt. % to 50 wt. %, e.g., from 0.2 wt. % to 25 wt. %, from 0.5 wt. % to 20 wt. %, or from 1 wt. % to 15 wt. %, based on the total weight of the catalyst. When the support modifier comprises tungsten, molybdenum, and vanadium, the support modifier may be present in an amount from 0.1 to 40 wt. %, e.g., from 0.1 to 30 wt. % or from 10 to 25 wt. %, based on the total weight of the catalyst. The support modifier may be substantially free of cobalt and active metals, such as tin.

As indicated, the support modifiers may adjust the acidity of the support. For example, the acid sites, e.g., Brønsted acid sites or Lewis acid sites, on the support material may be adjusted by the support modifier to favor selectivity to ethanol during the hydrogenation of acetic acid and/or esters thereof. The acidity of the support material may be adjusted by optimizing surface acidity of the support material. The support material may also be adjusted by having the support modifier change the pKa of the support material. Unless the context indicates otherwise, the acidity of a surface or the number of acid sites thereupon may be determined by the technique described in F. Delannay, Ed., “Characterization of Heterogeneous Catalysts”; Chapter III: Measurement of Acidity of Surfaces, p. 370-404; Marcel Dekker, Inc., N.Y. 1984, the entirety of which is incorporated herein by reference. In general, the surface acidity of the support may be adjusted based on the composition of the feed stream being sent to the hydrogenation process in order to maximize alcohol production, e.g., ethanol production.

In some embodiments, the support modifier may be an acidic modifier that increases the acidity of the catalyst. Suitable acidic support modifiers may be selected from the group consisting of: oxides of Group IVB metals, oxides of Group VB metals, oxides of Group VIB metals, oxides of Group VIIB metals, oxides of Group VIII metals, aluminum oxides, and mixtures thereof. In one embodiment, the support modifier comprises metal selected from the group consisting of tungsten, molybdenum, vanadium, niobium, and tantalum.

In one embodiment, the acidic modifier may also include those selected from the group consisting of WO₃, MoO₃, V₂O₅, V^{O}₂, V₂O₃, Nb₂O₅, Ta₂O₅, Al₂O₃, B₂O₃, P₂O₅, and Sb₂O₃, and Bi₂O₃. Reduced tungsten oxides or molybdenum oxides may also be employed, such as, for example, one or more of W₂₀O₅₈, WO₂, W₄₉O₁₁₉, W₅₀O₁₄₈, W₁₈O₄₉, Mo₉O₂₆, Mo₈O₂₃, Mo₅O₁₄, Mo₁₇O₄₇, Mo₄O₁₁, or MoO₂. In one embodiment, the tungsten oxide may be cubic or monoclinic tungsten oxide (H_{0.5}WO₃). It has now surprisingly and unexpectedly been discovered that the use of such metal oxide support modifiers in combination with a precious metal, cobalt, and one or more active metals may result in catalysts having multifunctionality, and which may be suitable for converting a carboxylic acid, such as acetic acid, as well as corresponding esters thereof, e.g., ethyl acetate, to one or more hydrogenation products, such as ethanol, under hydrogenation conditions.

In one embodiment, the catalyst comprises from 0.25 to 1.25 wt. % platinum, from 1 to 10 wt. % cobalt, and from 1 to 10 wt. % tin on a silica or a silica-alumina support material. The support material may comprise from 5 to 15 wt. % acidic support modifiers, such as H_{0.5}WO₃, WO₃, V₂O₅ and/or MoO₃.

### Processes for Making the Catalyst

The present invention also relates to processes for making the catalyst. Without being bound by theory, the process for making the catalyst may improve one or more of acetic acid conversion, ester conversion, ethanol selectivity and overall productivity. In one embodiment, the support is modified with one or more support modifiers and the resulting modified support is subsequently impregnated with cobalt, a precious metal and active metals to form the catalyst composition. For example, the support may be impregnated with a support modifier solution comprising a support modifier precursor and optionally one or more active metal precursors to form the modified support. After drying and calcination, the resulting modified support is impregnated with a second solution comprising precious metal precursor and optionally one or more of the active metal precursors, followed by drying and calcination to form the final catalyst.

In some embodiments, the support modifier may be added as particles to the support material. For example, one or more support modifier precursors, if desired, may be added to the support material by mixing the support modifier particles with the support material, preferably in water. When mixed it is preferred for some support modifiers to use a powdered material of the support modifiers. If a powdered material is employed, the support modifier may be pelletized, crushed and sieved prior to being added to the support.

As indicated, in most embodiments, the support modifier preferably is added through a wet impregnation step. Preferably, a support modifier precursor to the support modifier may be used. Some exemplary support modifier precursors include alkali metal oxides, alkaline earth metal oxides, Group IIB metal oxides, Group IIIB metal oxides, Group IVB metal oxides, Group VB metal oxides, Group VIB metal oxides, Group VIIB metal oxides, and/or Group VIII metal oxides, as well as preferably aqueous salts thereof.

Although the overwhelming majority of metal oxides and polyoxoion salts are insoluble, or have a poorly defined or limited solution chemistry, the class of isopoly- and heteropolyoxoanions of the early transition elements forms an important exception. These complexes may be represented by the general formulae:

[MₘO_{y}]^{p-} Isopolyanions

[XₓMₘO_{y}]^{q-} (x≦m) Heteropolyanions

where M is selected from tungsten, molybdenum, vanadium, niobium, tantalum and mixtures thereof, in their highest (d⁰, d¹) oxidations states. Such polyoxometalate anions form a structurally distinct class of complexes based predominately, although not exclusively, upon quasi-octahedrally-coordinated metal atoms. The elements that can function as the addenda atoms, M, in heteropoly- or isopolyanions may be limited to those with both a favorable combination of ionic radius and charge and the ability to form d_{π}-p_{π}M—O bonds. There is little restriction, however, on the heteroatom, X, which may be selected from virtually any element other than the rare gases. See, e.g., M. T. Pope, Heteropoly and Isopoly Oxometalates, Springer Verlag, Berlin, 1983, 180; Chapt. 38, Comprehensive Coordination Chemistry, Vol. 3, 1028-58, Pergamon Press, Oxford, 1987, the entireties of which are incorporated herein by reference.

Polyoxometalates (POMs) and their corresponding heteropoly acids (HPAs) have several advantages making them economically and environmentally attractive. First, HPAs have a very strong approaching the superacid region, Bronsted acidity. In addition, they are efficient oxidants exhibiting fast reversible multielectron redox transformations under rather mild conditions. Solid HPAs also possess a discrete ionic structure, comprising fairly mobile basic structural units, e.g., heteropolyanions and countercations (H⁺, H₃O⁺, H₅O₂⁺, etc.), unlike zeolites and metal oxides.

In view of the foregoing, in some embodiments, the support modifier precursor comprises a POM, which preferably comprises a metal selected from the group consisting of tungsten, molybdenum, niobium, vanadium and tantalum. In some embodiments, the POM comprises a hetero-POM. A non-limiting list of suitable POMs includes phosphotungstic acid (H—PW₁₂)(H₃PW₁₂O₄₀.nH₂O), ammonium metatungstate (AMT) ((NH₄)₆H₂W₁₂O₄₀.H₂O), ammonium heptamolybdate tetrahydrate, (AHM) ((NH₄)₆Mo₇O₂₄.4H₂O), silicotungstic acid hydrate (H—SiW₁₂)(H₄SiW₁₂O₄₀.H₂O), silicomolybdic acid (H—SiMo₁₂)(H₄SiMo₁₂O₄₀.nH₂O), and phosphomolybdic acid (H-PMo₁₂)(H₃PMo₁₂O₄₀.nH₂O).

The use of POM-derived support modifiers in the catalyst compositions of the invention has now surprising and unexpectedly been shown to provide bi- or multi-functional catalyst functionality, desirably resulting in conversions for both acetic acid and byproduct esters such as ethyl acetate, thereby rendering them suitable for catalyzing mixed feeds comprising, for example, acetic acid and ethyl acetate.

Impregnation of the cobalt, precious metal and one or more active metals onto the support, e.g., modified support, may occur simultaneously (co-impregnation) or sequentially. In simultaneous impregnation, the two or more metal precursors are mixed together and added to the support, preferably modified support, together followed by drying and calcination to form the final catalyst composition. With simultaneous impregnation, it may be desired to employ a dispersion agent, surfactant, or solubilizing agent, e.g., ammonium oxalate or an acid such as acetic or nitric acid, to facilitate the dispersing or solubilizing of the first, second and/or optional third metal precursors in the event the two precursors are incompatible with the desired solvent, e.g., water.

In sequential impregnation, the first metal precursor may be first added to the support followed by drying and calcining, and the resulting material may then be impregnated with the second metal precursor followed by an additional drying step followed by a calcining step to form the final catalyst composition. Additional metal precursors (e.g., a third metal precursor) may be added either with the first and/or second metal precursor or in a separate third impregnation step, followed by drying and calcination. Of course, combinations of sequential and simultaneous impregnation may be employed if desired.

The use of a solvent, such as water, glacial acetic acid, a strong acid such as hydrochloric acid, nitric acid, or sulfuric acid, or an organic solvent, is preferred in the support modification step, e.g., for impregnating a support modifier precursor onto the support material. The support modifier solution comprises the solvent, preferably water, a support modifier precursor, and preferably one or more active metal precursors. The solution is stirred and combined with the support material using, for example, incipient wetness techniques in which the support modifier precursor is added to a support material having the same pore volume as the volume of the solution. Impregnation occurs by adding, optionally drop wise, a solution containing the precursors of either or both the support modifiers and/or active metals, to the dry support material. Capillary action then draws the support modifier into the pores of the support material. The thereby impregnated support can then be formed by drying, optionally under vacuum, to drive off solvents and any volatile components within the support mixture and depositing the support modifier on and/or within the support material. Drying may occur, for example, at a temperature of from 50° C. to 300° C., e.g., from 100° C. to 200° C. or about 120° C., optionally for a period of from 1 to 24 hours, e.g., from 3 to 15 hours or from 6 to 12 hours. The dried support may be calcined optionally with ramped heating, for example, at a temperature from 300° C. to 900° C., e.g., from 400° C. to 750° C., from 500° C. to 600° C. or at about 550° C., optionally for a period of time from 1 to 12 hours, e.g., from 2 to 10 hours, from 4 to 8 hours or about 6 hours, to form the final modified support. Upon heating and/or the application of vacuum, the metal(s) of the precursor(s) preferably decompose into their oxide or elemental form. In some cases, the completion of removal of the solvent may not take place until the catalyst is placed into use and/or calcined, e.g., subjected to the high temperatures encountered during operation. During the calcination step, or at least during the initial phase of use of the catalyst, such compounds are converted into a catalytically active form of the metal or a catalytically active oxide thereof.

Once formed, the modified supports may be shaped into particles having the desired size distribution, e.g., to form particles having an average particle size in the range of from 0.2 to 0.4 cm. The supports may be extruded, pelletized, tabletized, pressed, crushed or sieved to the desired size distribution. Any of the known methods to shape the support materials into desired size distribution can be employed. Alternatively, support pellets may be used as the starting material used to make the modified support and, ultimately, the final catalyst.

In one embodiment, the catalyst of the present invention may be prepared using a bulk catalyst technique. Bulk catalysts may be formed by precipitating precursors to support modifiers and one or more active metals. The precipitating may be controlled by changing the temperature, pressure, and/or pH. In some embodiments, the bulk catalyst preparation may use a binder. A support material may not be used in a bulk catalyst process. Once precipitated, the bulk catalyst may be shaped by spraying drying, pelleting, granulating, tablet pressing, beading, or pilling. Suitable bulk catalyst techniques may be used such as those described in Krijn P. de Jong, ed., Synthesis of Solid Catalysts, Wiley, (2009), pg. 308, the entire contents and disclosure of which is incorporated by reference.

In one embodiment, cobalt, a precious metal and one or more active metals are impregnated onto the support, preferably onto any of the above-described modified supports. A precursor of the precious metal preferably is used in the metal impregnation step, such as a water soluble compound or water dispersible compound/complex that includes the precious metal of interest. Similarly, precursors to cobalt and one or more active metals may also be impregnated into the support, preferably modified support. Depending on the metal precursors employed, the use of a solvent, such as water, glacial acetic acid, nitric acid or an organic solvent, may be preferred to help solubilize one or more of the metal precursors.

In one embodiment, separate solutions of the metal precursors are formed, which are subsequently blended prior to being impregnated on the support. For example, a first solution may be formed comprising a first metal precursor, and a second solution may be formed comprising the second metal precursor and optionally the third metal precursor. At least one of the metal precursors is a cobalt precursor, and preferably another metal precursor is a precious metal precursor, and the other(s) are preferably active metal precursors. Either or both solutions preferably comprise a solvent, such as water, glacial acetic acid, hydrochloric acid, nitric acid or an organic solvent.

In one exemplary embodiment, a first solution comprising a first metal halide is prepared. The first metal halide optionally comprises a tin halide, e.g., a tin chloride such as tin (II) chloride and/or tin (IV) chloride. Optionally, a second metal precursor, as a solid or as a separate solution, is combined with the first solution to form a combined solution. The second metal precursor, if used, preferably comprises a second metal oxalate, acetate, halide or nitrate, e.g., cobalt nitrate. The first metal precursor comprises cobalt, and the second metal precursor comprises another active metal, such as copper, iron, nickel, titanium, zinc, chromium, tin, lanthanum, cerium, and manganese. A second solution is also prepared comprising a precious metal precursor, in this embodiment preferably a precious metal halide, such as a halide of rhodium, rhenium, ruthenium, platinum or palladium. The second solution is combined with the first solution or the combined solution, depending on whether the second metal precursor is desired, to form a mixed metal precursor solution. The resulting mixed metal precursor solution may then be added to the support, optionally a modified support, followed by drying and calcining to form the final catalyst composition as described above. The resulting catalyst may or may not be washed after the final calcination step. Due to the difficulty in solubilizing some precursors, it may be desired to reduce the pH of the first and/or second solutions, for example by employing an acid such as acetic acid, hydrochloric acid or nitric acid, e.g., 6-10 M HNO₃.

In another aspect, a first solution comprising a first metal oxalate is prepared, such as an oxalate of cobalt, copper, iron, nickel, titanium, zinc, chromium, tin, lanthanum, cerium, and manganese. In this embodiment, the first solution preferably further comprises an acid such as acetic acid, hydrochloric acid, phosphoric acid or nitric acid, e.g., 6-10 M HNO₃. Optionally, a second metal precursor, as a solid or as a separate solution, is combined with the first solution to form a combined solution. The second metal precursor, if used, preferably comprises a second metal oxalate, acetate, halide or nitrate, and preferably comprises an active metal, also optionally cobalt, copper, iron, nickel, titanium, zinc, chromium, tin, lanthanum, cerium, and manganese. A second solution is also formed comprising a precious metal oxalate, for example, an oxalate of rhodium, rhenium, ruthenium, platinum or palladium, and optionally further comprises an acid such as acetic acid, hydrochloric acid, phosphoric acid or nitric acid, e.g., 6-10 M HNO₃. The second solution is combined with the first solution or the combined solution, depending on whether the second metal precursor is desired, to form a mixed metal precursor solution. The resulting mixed metal precursor solution may then be added to the support, optionally a modified support, followed by drying and calcining to form the final catalyst composition as described above. The resulting catalyst may or may not be washed after the final calcination step.

In one embodiment, the impregnated support, optionally impregnated modified support, is dried at a temperature from 100° C. to 140° C., from 110° C. to 130° C., or about 120° C., optionally from 1 to 12 hours, e.g., from 2 to 10 hours, from 4 to 8 hours or about 6 hours. If calcination is desired, it is preferred that the calcination temperature employed in this step is less than the calcination temperature employed in the formation of the modified support, discussed above. The second calcination step, for example, may be conducted at a temperature that is at least 50° C., at least 100° C., at least 150° C. or at least 200° C. less than the first calcination step, i.e., the calcination step used to form the modified support. For example, the impregnated catalyst may be calcined at a temperature from 200° C. to 500° C., from 300° C. to 400° C., or about 350° C., optionally for a period of from 1 to 12 hours, e.g., from 2 to 10 hours, from 4 to 8 hours or about 6 hours.

In one embodiment, ammonium oxalate is used to facilitate solubilizing of at least one of the metal precursors, e.g., a tin precursor, as described in U.S. Pat. No. 8,211,821, the entirety of which is incorporated herein by reference. In this aspect, the first metal precursor optionally comprises an oxalate of a precious metal, e.g., rhodium, palladium, or platinum, and a second metal precursor optionally comprises an oxalate tin. A cobalt metal precursor comprises a nitrate, halide, acetate or oxalate. In this aspect, a solution of the second metal precursor may be made in the presence of ammonium oxalate as solubilizing agent, and the first metal precursor may be added thereto, optionally as a solid or a separate solution. If used, the third metal precursor may be combined with the solution comprising the first and second metal precursors, or may be combined with the second metal precursor, optionally as a solid or a separate solution, prior to addition of the first metal precursor. In other embodiments, an acid such as acetic acid, hydrochloric acid or nitric acid may be substituted for the ammonium oxalate to facilitate solubilizing of the tin oxalate. The resulting mixed metal precursor solution may then be added to the support, optionally a modified support, followed by drying and calcining to form the final catalyst composition as described above.

The specific precursors used in the various embodiments of the invention may vary widely. Suitable metal precursors may include, for example, metal halides, amine solubilized metal hydroxides, metal nitrates or metal oxalates. For example, suitable compounds for platinum precursors and palladium precursors include chloroplatinic acid, ammonium chloroplatinate, amine solubilized platinum hydroxide, platinum nitrate, platinum tetra ammonium nitrate, platinum chloride, platinum oxalate, palladium nitrate, palladium tetra ammonium nitrate, palladium chloride, palladium oxalate, sodium palladium chloride, sodium platinum chloride, and platinum ammonium nitrate, Pt(NH₃)₄(NO₄)₂. Generally, both from the point of view of economics and environmental aspects, aqueous solutions of soluble compounds of platinum and palladium are preferred. In one embodiment, the precious metal precursor is not a metal halide and is substantially free of metal halides, while in other embodiments, as described above, the precious metal precursor is a halide.

As another example, PtSnCo/WO₃ on SiO₂ may be prepared by first impregnating a precursor to WO₃, preferably a POM precursor to WO₃, on the SiO₂, followed by the co-impregnation with chloroplatinic acid, tin (IV) chloride, and cobalt nitrate. Again, each impregnation step may be followed by drying and calcination steps, with the second calcination temperature preferably being less than the first calcination temperature. The resulting modified support may be impregnated, preferably in a single impregnation step, with one or more of the first, second and third metals, including cobalt, followed by a second drying and calcination step. Optionally, cobalt tungstate may be formed on the modified support. The support modifier does not comprise tin tungstate, even though the support modifier may comprise tin. Again, the temperature of the second calcining step preferably is less than the temperature of the first calcining step.

### Use of Catalyst to Hydrogenate Acetic Acid

One advantage of catalysts of the present invention is the stability or activity of the catalyst for producing ethanol. Accordingly, it can be appreciated that the catalysts of the present invention are fully capable of being used in commercial scale industrial applications for hydrogenation of acetic acid, particularly in the production of ethanol. In particular, it is possible to achieve such a degree of stability such that catalyst activity will have a rate of productivity decline that is less than 6% per 100 hours of catalyst usage, e.g., less than 3% per 100 hours or less than 1.5% per 100 hours. Preferably, the rate of productivity decline is determined once the catalyst has achieved steady-state conditions.

After the washing, drying and calcining of the catalyst is completed, the catalyst may be reduced in order to activate it. Reduction is carried out in the presence of a reducing gas, preferably hydrogen. The reducing gas is optionally continuously passed over the catalyst at an initial ambient temperature that is increased up to 400° C. In one embodiment, the reduction is carried out after the catalyst has been loaded into the reaction vessel where the hydrogenation will be carried out.

In one embodiment the invention is to a process for producing ethanol by hydrogenating a feed stream comprising compounds selected from acetic acid, ethyl acetate and mixtures thereof in the presence of any of the above-described catalysts. One particular preferred reaction is to make ethanol from acetic acid. The hydrogenation reaction may be represented as follows:

HOAc+2H₂→EtOH+H₂O

In some embodiments, the catalyst may be characterized as a bifunctional catalyst in that it effectively catalyzes the hydrogenation of acetic acid to ethanol as well as the conversion of ethyl acetate to one or more products, preferably ethanol.

The raw materials, acetic acid and hydrogen, fed to the reactor used in connection with the process of this invention may be derived from any suitable source including natural gas, petroleum, coal, biomass, and so forth. As examples, acetic acid may be produced via methanol carbonylation, acetaldehyde oxidation, ethane oxidation, oxidative fermentation, and anaerobic fermentation. Methanol carbonylation processes suitable for production of acetic acid are described in U.S. Pat. Nos. 7,208,624; 7,115,772; 7,005,541; 6,657,078; 6,627,770; 6,143,930; 5,599,976; 5,144,068; 5,026,908; 5,001,259; and 4,994,608, the entire disclosures of which are incorporated herein by reference. Optionally, the production of ethanol may be integrated with such methanol carbonylation processes.

As petroleum and natural gas prices fluctuate becoming either more or less expensive, methods for producing acetic acid and intermediates such as methanol and carbon monoxide from other carbon sources have drawn increasing interest. In particular, when petroleum is relatively expensive, it may become advantageous to produce acetic acid from synthesis gas (“syngas”) that is derived from other available carbon sources. U.S. Pat. No. 6,232,352, the entirety of which is incorporated herein by reference, for example, teaches a method of retrofitting a methanol plant for the manufacture of acetic acid. By retrofitting a methanol plant, the large capital costs associated with CO generation for a new acetic acid plant are significantly reduced or largely eliminated. All or part of the syngas is diverted from the methanol synthesis loop and supplied to a separator unit to recover CO, which is then used to produce acetic acid. In a similar manner, hydrogen for the hydrogenation step may be supplied from syngas.

In some embodiments, some or all of the raw materials for the above-described acetic acid hydrogenation process may be derived partially or entirely from syngas. For example, the acetic acid may be formed from methanol and carbon monoxide, both of which may be derived from syngas. The syngas may be formed by partial oxidation reforming or steam reforming, and the carbon monoxide may be separated from syngas. Similarly, hydrogen that is used in the step of hydrogenating the acetic acid to form the crude ethanol product may be separated from syngas. The syngas, in turn, may be derived from variety of carbon sources. The carbon source, for example, may be selected from the group consisting of natural gas, oil, petroleum, coal, biomass, and combinations thereof. Syngas or hydrogen may also be obtained from bio-derived methane gas, such as bio-derived methane gas produced by landfills or agricultural waste.

Biomass-derived syngas has a detectable ¹⁴C isotope content as compared to fossil fuels such as coal or natural gas. An equilibrium forms in the Earth's atmosphere between constant new formation and constant degradation, and so the proportion of the ¹⁴C nuclei in the carbon in the atmosphere on Earth is constant over long periods. The same distribution ratio n¹⁴C:n¹²C ratio is established in living organisms as is present in the surrounding atmosphere, which stops at death and ¹⁴C decomposes at a half life of about 6000 years. Methanol, acetic acid and/or ethanol formed from biomass-derived syngas would be expected to have a ¹⁴C content that is substantially similar to living organisms. For example, the ¹⁴C:¹²C ratio of the methanol, acetic acid and/or ethanol may be from one half to about 1 of the ¹⁴C:¹²C ratio for living organisms. In other embodiments, the syngas, methanol, acetic acid and/or ethanol described herein are derived wholly from fossil fuels, i.e. carbon sources produced over 60,000 years ago, may have no detectable ¹⁴C content.

In another embodiment, the acetic acid used in the hydrogenation step may be formed from the fermentation of biomass. The fermentation process preferably utilizes an acetogenic process or a homoacetogenic microorganism to ferment sugars to acetic acid producing little, if any, carbon dioxide as a by-product. The carbon efficiency for the fermentation process preferably is greater than 70%, greater than 80% or greater than 90% as compared to conventional yeast processing, which typically has a carbon efficiency of about 67%. Optionally, the microorganism employed in the fermentation process is of a genus selected from the group consisting of Clostridium, Lactobacillus, Moorella, Thermoanaerobacter, Propionibacterium, Propionispera, Anaerobiospirillum, and Bacteriodes, and in particular, species selected from the group consisting of Clostridium formicoaceticum, Clostridium butyricum, Moorella thermoacetica, Thermoanaerobacter kivui, Lactobacillus delbrukii, Propionibacterium acidipropionici, Propionispera arboris, Anaerobiospirillum succinicproducens, Bacteriodes amylophilus and Bacteriodes ruminicola. Optionally, in this process, all or a portion of the unfermented residue from the biomass, e.g., lignans, may be gasified to form hydrogen that may be used in the hydrogenation step of the present invention. Exemplary fermentation processes for forming acetic acid are disclosed in U.S. Pat. No. 6,509,180, and U.S. Pub. Nos. 2008/0193989 and 2009/0281354, the entireties of which are incorporated herein by reference.

Examples of biomass include, but are not limited to, agricultural wastes, forest products, grasses, and other cellulosic material, timber harvesting residues, softwood chips, hardwood chips, tree branches, tree stumps, leaves, bark, sawdust, off-spec paper pulp, corn, corn stover, wheat straw, rice straw, sugarcane bagasse, switchgrass, miscanthus, animal manure, municipal garbage, municipal sewage, commercial waste, grape pumice, almond shells, pecan shells, coconut shells, coffee grounds, grass pellets, hay pellets, wood pellets, cardboard, paper, plastic, and cloth. Another biomass source is black liquor, which is an aqueous solution of lignin residues, hemicellulose, and inorganic chemicals.

U.S. Pat. No. RE 35,377, also incorporated herein by reference, provides a method for the production of methanol by conversion of carbonaceous materials such as oil, coal, natural gas and biomass materials. The process includes hydrogasification of solid and/or liquid carbonaceous materials to obtain a process gas which is steam pyrolized with additional natural gas to form syngas. The syngas is converted to methanol which may be carbonylated to acetic acid. The method likewise produces hydrogen which may be used in connection with this invention as noted above. U.S. Pat. No. 5,821,111, which discloses a process for converting waste biomass through gasification into syngas, and U.S. Pat. No. 6,685,754, which discloses a method for the production of a hydrogen-containing gas composition, such as a syngas including hydrogen and carbon monoxide, are incorporated herein by reference in their entireties.

The acetic acid fed to the hydrogenation reactor may also comprise other carboxylic acids and anhydrides, as well as aldehyde and/or ketones, such as acetaldehyde and acetone. Preferably, the feed stream comprises acetic acid and ethyl acetate. A suitable acetic acid feed stream comprises one or more of the compounds selected from the group consisting of acetic acid, acetic anhydride, acetaldehyde, ethyl acetate, diethyl acetal, diethyl ether, and mixtures thereof. These other compounds may also be hydrogenated in the processes of the present invention. In some embodiments, the presence of carboxylic acids, such as propanoic acid or its aldehyde, may be beneficial in producing propanol. Water may also be present in the acetic acid feed.

Alternatively, acetic acid in vapor form may be taken directly as crude product from the flash vessel of a methanol carbonylation unit of the class described in U.S. Pat. No. 6,657,078, the entirety of which is incorporated herein by reference. The crude vapor product, for example, may be fed directly to the hydrogenation reactor without the need for condensing the acetic acid and light ends or removing water, saving overall processing costs.

The acetic acid may be vaporized at the reaction temperature, following which the vaporized acetic acid may be fed along with hydrogen in an undiluted state or diluted with a relatively inert carrier gas, such as nitrogen, argon, helium, carbon dioxide and the like. For reactions run in the vapor phase, the temperature should be controlled in the system such that it does not fall below the dew point of acetic acid. In one embodiment, the acetic acid may be vaporized at the boiling point of acetic acid at the particular pressure, and then the vaporized acetic acid may be further heated to the reactor inlet temperature. In another embodiment, the acetic acid is mixed with other gases before vaporizing, followed by heating the mixed vapors up to the reactor inlet temperature. Preferably, the acetic acid is transferred to the vapor state by passing hydrogen and/or recycle gas through the acetic acid at a temperature at or below 125° C., followed by heating of the combined gaseous stream to the reactor inlet temperature.

The reactor, in some embodiments, may include a variety of configurations using a fixed bed reactor or a fluidized bed reactor. In many embodiments of the present invention, an “adiabatic” reactor can be used; that is, there is little or no need for internal plumbing through the reaction zone to add or remove heat. In other embodiments, a radial flow reactor or reactors may be employed as the reactor, or a series of reactors may be employed with or without heat exchange, quenching, or introduction of additional feed material. Alternatively, a shell and tube reactor provided with a heat transfer medium may be used. In many cases, the reaction zone may be housed in a single vessel or in a series of vessels with heat exchangers therebetween.

In preferred embodiments, the catalyst is employed in a fixed bed reactor, e.g., in the shape of a pipe or tube, where the reactants, typically in the vapor form, are passed over or through the catalyst. Other reactors, such as fluid or ebullient bed reactors, can be employed. In some instances, the hydrogenation catalysts may be used in conjunction with an inert material to regulate the pressure drop of the reactant stream through the catalyst bed and the contact time of the reactant compounds with the catalyst particles. In some embodiments, multiple catalyst beds are employed in the same reactor or in different reactors, e.g., in series. For example, in one embodiment, a first catalyst functions in a first catalyst stage as a catalyst for the hydrogenation of a carboxylic acid, e.g., acetic acid, to its corresponding alcohol, e.g., ethanol, and a second bifunctional catalyst is employed in the second stage for converting unreacted acetic acid to ethanol as well as converting byproduct ester, e.g., ethyl acetate, to additional products, preferably to ethanol. The catalysts of the invention may be employed in either or both the first and/or second stages of such reaction systems.

The hydrogenation in the reactor may be carried out in either the liquid phase or vapor phase. Preferably, the reaction is carried out in the vapor phase under the following conditions. The reaction temperature may range from 125° C. to 350° C., e.g., from 200° C. to 325° C., from 225° C. to 300° C., or from 250° C. to 300° C. The pressure may range from 10 kPa to 3000 kPa, e.g., from 50 kPa to 2300 kPa, or from 100 kPa to 2000 kPa. The reactants may be fed to the reactor at a gas hourly space velocity (GHSV) of greater than 500 hr⁻¹, e.g., greater than 1000 hr⁻¹, greater than 2500 hr⁻¹ or even greater than 5000 hr⁻¹. In terms of ranges the GHSV may range from 50 hr⁻¹ to 50,000 hr⁻¹, e.g., from 500 hr⁻¹ to 30,000 hr⁻¹, from 1000 hr⁻¹ to 10,000 hr⁻¹, or from 1000 hr⁻¹ to 6500 hr⁻¹.

The hydrogenation optionally is carried out at a pressure just sufficient to overcome the pressure drop across the catalytic bed at the GHSV selected, although there is no bar to the use of higher pressures, it being understood that considerable pressure drop through the reactor bed may be experienced at high space velocities, e.g., 5000 hr⁻¹ or 6,500 hr⁻¹.

Although the reaction consumes two moles of hydrogen per mole of acetic acid to produce one mole of ethanol, the actual molar ratio of hydrogen to acetic acid in the feed stream may vary from about 100:1 to 1:100, e.g., from 50:1 to 1:50, from 20:1 to 1:2, or from 18:1 to 2:1. Most preferably, the molar ratio of hydrogen to acetic acid is greater than 2:1, e.g., greater than 4:1 or greater than 8:1. For a mixed feed stream, the molar ratio of hydrogen to ethyl acetate may be greater than 5:1, e.g., greater than 10:1 or greater than 15:1.

Contact or residence time can also vary widely, depending upon such variables as amount of feed stream (acetic acid and/or ethyl acetate), catalyst, reactor, temperature, and pressure. Typical contact times range from a fraction of a second to more than several hours when a catalyst system other than a fixed bed is used, with preferred contact times, at least for vapor phase reactions, from 0.1 to 100 seconds, e.g., from 0.3 to 80 seconds or from 0.4 to 30 seconds.

In particular, by employing the catalysts of the invention, the hydrogenation of acetic acid and/or ethyl acetate may achieve favorable conversion and favorable selectivity and productivity to ethanol in the reactor. For purposes of the present invention, the term “conversion” refers to the amount of acetic acid or ethyl acetate, whichever is specified, in the feed that is converted to a compound other than acetic acid or ethyl acetate, respectively. Conversion is expressed as a percentage based on acetic acid or ethyl acetate in the feed. The acetic acid conversion may be at least 20%, more preferably at least 60%, at least 75%, at least 80%, at least 90%, at least 95% or at least 99%.

During the hydrogenation of acetic acid, ethyl acetate may be produced as a byproduct. Without consuming any ethyl acetate from the mixed vapor phase reactants, the conversion of ethyl acetate would be deemed negative. Some of the catalysts described herein are monofunctional in nature and are effective for converting acetic acid to ethanol, but not for converting ethyl acetate. The use of monofunctional catalysts may result in the undesirable build up of ethyl acetate in the system, particularly for systems employing one or more recycle streams that contain ethyl acetate to the reactor.

The preferred catalysts of the invention, however, are multifunctional in that they effectively catalyze the conversion of acetic acid to ethanol as well as the conversion of an alkyl acetate, such as ethyl acetate, to one or more products other than that alkyl acetate. The multifunctional catalyst is preferably effective for consuming ethyl acetate at a rate sufficiently great so as to at least offset the rate of ethyl acetate production, thereby resulting in a non-negative ethyl acetate conversion, i.e., no net increase in ethyl acetate is realized. The use of such catalysts may result, for example, in an ethyl acetate conversion that is effectively 0% or that is greater than 0%. In some embodiments, the catalysts of the invention are effective in providing ethyl acetate conversions of at least 0%, e.g., at least 5%, at least 10%, at least 15%, at least 20%, or at least 35%.

In continuous processes, the ethyl acetate being added (e.g., recycled) to the hydrogenation reactor and ethyl acetate leaving the reactor in the crude product preferably approaches a certain level after the process reaches equilibrium. The use of a multifunctional catalyst that catalyzes the conversion of ethyl acetate as well as acetic acid results in a lower amount of ethyl acetate added to the reactor and less ethyl acetate produced relative to monofunctional catalysts. In preferred embodiments, the concentration of ethyl acetate in the mixed feed and crude product is less than 40 wt. %, less than 25 wt. % or less than 15 wt. %, after equilibrium has been achieved. In preferred embodiments, the process forms a crude product comprising ethanol and ethyl acetate, and the crude product has an ethyl acetate steady state concentration from 0.1 to 40 wt. %, e.g., from 0.1 to 20 wt. % or from 0.1 to 15 wt. %.

Although catalysts that have high acetic acid conversions are desirable, such as at least 60%, in some embodiments a low conversion may be acceptable at high selectivity for ethanol. It is, of course, well understood that in many cases, it is possible to compensate for conversion by appropriate recycle streams or use of larger reactors, but it is more difficult to compensate for poor selectivity.

Selectivity is expressed as a mole percent based on converted acetic acid and/or ethyl acetate. It should be understood that each compound converted from acetic acid and/or ethyl acetate has an independent selectivity and that selectivity is independent of conversion. For example, if 60 mole % of the converted acetic acid is converted to ethanol, we refer to the ethanol selectivity as 60%. For purposes of the present invention, the total selectivity is based on the combined converted acetic acid and ethyl acetate. Preferably, total selectivity to ethanol is at least 60%, e.g., at least 70%, or at least 80%, at least 85% or at least 88%. Preferred embodiments of the hydrogenation process also have low selectivity to undesirable products, such as methane, ethane, and carbon dioxide. The selectivity to these undesirable products preferably is less than 4%, e.g., less than 2% or less than 1%. More preferably, these undesirable products are present in undetectable amounts. Formation of alkanes may be low, and ideally less than 2%, less than 1%, or less than 0.5% of the acetic acid passed over the catalyst is converted to alkanes, which have little value other than as fuel.

The term “productivity,” as used herein, refers to the grams of a specified product, e.g., ethanol, formed during the hydrogenation based on the kilograms of catalyst used per hour. A productivity of at least 100 grams of ethanol per kilogram of catalyst per hour, e.g., at least 400 grams of ethanol per kilogram of catalyst per hour or at least 600 grams of ethanol per kilogram of catalyst per hour, is preferred. In terms of ranges, the productivity preferably is from 100 to 3,000 grams of ethanol per kilogram of catalyst per hour, e.g., from 400 to 2,500 grams of ethanol per kilogram of catalyst per hour or from 600 to 2,000 grams of ethanol per kilogram of catalyst per hour.

In various embodiments of the present invention, the crude ethanol product produced by the reactor, before any subsequent processing, such as purification and separation, will typically comprise unreacted acetic acid, ethanol and water. Exemplary compositional ranges for the crude ethanol product are provided in Table 1. The “others” identified in Table 1 may include, for example, esters, ethers, aldehydes, ketones, alkanes, and carbon dioxide.

TABLE 1
CRUDE ETHANOL PRODUCT COMPOSITIONS

| | Conc. | Conc. | Conc. | Conc. |
|---|---|---|---|---|
| Component | (wt. %) | (wt. %) | (wt. %) | (wt. %) |
| Ethanol | 5 to 72 | 15 to 72 | 15 to 70 | 25 to 65 |
| Acetic Acid | 0 to 90 | 0 to 50 | 0 to 35 | 0 to 15 |
| Water | 5 to 40 | 5 to 30 | 10 to 30 | 10 to 26 |
| Ethyl Acetate | 0 to 30 | 1 to 25 | 3 to 20 | 5 to 18 |
| Acetaldehyde | 0 to 10 | 0 to 3 | 0.1 to 3 | 0.2 to 2 |
| Others | 0.1 to 10 | 0.1 to 6 | 0.1 to 4 | — |

In one embodiment, the crude ethanol product may comprise acetic acid in an amount less than 20 wt. %, e.g., of less than 15 wt. %, less than 10 wt. % or less than 5 wt. %. In terms of ranges, the acetic acid concentration of Table 1 may range from 0.1 wt. % to 20 wt. %, e.g., 0.1 wt. % to 15 wt. %, from 0.1 wt. % to 10 wt. % or from 0.1 wt. % to 5 wt. %. In embodiments having lower amounts of acetic acid, the conversion of acetic acid is preferably greater than 75%, e.g., greater than 85% or greater than 90%. In addition, the selectivity to ethanol may also be preferably high, and is greater than 75%, e.g., greater than 85% or greater than 90%.

An ethanol product may be recovered from the crude ethanol product produced by the reactor using the catalyst of the present invention may be recovered using several different techniques.

The ethanol product may be an industrial grade ethanol comprising from 75 to 96 wt. % ethanol, e.g., from 80 to 96 wt. % or from 85 to 96 wt. % ethanol, based on the total weight of the ethanol product. The industrial grade ethanol may have a water concentration of less than 12 wt. % water, e.g., less than 8 wt. % or less than 3 wt. %. In some embodiments, when further water separation is used, the ethanol product preferably contains ethanol in an amount that is greater than 96 wt. %, e.g., greater than 98 wt. % or greater than 99.5 wt. %. The ethanol product having further water separation preferably comprises less than 3 wt. % water, e.g., less than 2 wt. % or less than 0.5 wt. %.

The finished ethanol composition produced by the embodiments of the present invention may be used in a variety of applications including fuels, solvents, chemical feedstocks, pharmaceutical products, cleansers, sanitizers, hydrogen transport or consumption. In fuel applications, the finished ethanol composition may be blended with gasoline for motor vehicles such as automobiles, boats and small piston engine aircraft. In non-fuel applications, the finished ethanol composition may be used as a solvent for toiletry and cosmetic preparations, detergents, disinfectants, coatings, inks, and pharmaceuticals. The finished ethanol composition may also be used as a processing solvent in manufacturing processes for medicinal products, food preparations, dyes, photochemicals and latex processing.

The finished ethanol composition may also be used as a chemical feedstock to make other chemicals such as vinegar, ethyl acrylate, ethyl acetate, ethylene, glycol ethers, ethylamines, ethyl benzene, aldehydes, butadiene, and higher alcohols, especially butanol. In the production of ethyl acetate, the finished ethanol composition may be esterified with acetic acid. In another application, the finished ethanol composition may be dehydrated to produce ethylene. Any known dehydration catalyst, such as zeolite catalysts or phosphotungstic acid calaysts, can be employed to dehydrate ethanol, as described in U.S. Pub. Nos. 2010/0030002 and 2010/0030001 and WO2010146332, the entire contents and disclosures of which are hereby incorporated by reference.

### Catalyst Regeneration

The catalysts of the invention are particularly robust and have long catalyst lifetimes. Nevertheless, over periods of extended usage, the activity of the catalysts of the invention may gradually be reduced. Accordingly, in another embodiment of the invention, the invention relates to a process for regenerating a spent hydrogenation catalyst, comprising contacting a carboxylic acid and hydrogen in a hydrogenation reactor with a hydrogenation catalyst under conditions effective to form a hydrogenation product and the spent hydrogenation catalyst; and treating the spent hydrogenation catalyst with a regenerating medium at a temperature greater than 200° C., optionally from 300° C. to 600° C., under conditions effective to form a regenerated hydrogenation catalyst having greater catalytic activity than the spent hydrogenation catalyst, wherein the hydrogenation catalyst comprises a precious metal and one or more active metals on a support. In this context, by “spent” it is meant a catalyst having reduced conversion and/or reduced selectivity for the desired product, e.g., ethanol, relative to an earlier usage period for the same catalyst, wherein the reduced selectivity and/or conversion cannot be recovered by increasing reactor temperature up to designed limits.

In another embodiment, the invention is to a process for regenerating a spent catalyst comprising (a) contacting a carboxylic acid and hydrogen in a hydrogenation reactor with a hydrogenation catalyst under conditions effective to form a hydrogenation product and the spent hydrogenation catalyst; and (b) treating the spent hydrogenation catalyst with a regenerating medium at a temperature greater than 200° C., optionally from 300° C. to 600° C., under conditions effective to form a regenerated hydrogenation catalyst having greater catalytic activity than the spent hydrogenation catalyst, wherein the hydrogenation catalyst comprises a precious metal and one or more active metals on a support. The treating may occur within the hydrogenation reactor, or external to the hydrogenation reactor. For example, the treating may occur in a regeneration unit, in which case the process further comprises the steps of directing the spent hydrogenation catalyst from the hydrogenation reactor to the regeneration unit, and directing the regenerated hydrogenation catalyst from the regeneration unit to the hydrogenation reactor.

The regenerating medium may vary depending on whether it is desired to merely “strip” the catalyst, for example of carbonaceous materials, or whether full regeneration is desired. Depending on the condition of the spent catalyst, the regenerating medium may be selected from steam, oxygen (optionally in the form of air, diluted air or an oxygen/nitrogen mixture optionally with variable O₂/N₂ ratio during regeneration treatment), or hydrogen. Preferably, the regeneration medium is substantially free of the carboxylic acid reactant, optionally comprising less than 10 wt. % carboxylic acids, less than 5 wt. % carboxylic acids, or less than 1 wt. % carboxylic acids, e.g., acetic acid. The treating step may occur, for example, at a pressure ranging from 0.5 to 10 bar, e.g., from 0.8 to 8 bar or from 0.9 to 4 bar. The regenerating may occur, for example, over a period ranging from 10 to 200 hours, e.g., from 20 to 150 hours or from 50 to 100 hours. Preferably, the conditions employed in the treating step are sufficient to increase the carboxylic acid conversion, e.g., acetic acid conversion, and/or ethanol selectivity of the resulting regenerated hydrogenation catalyst by at least 25%, e.g., at least 50%, or at least 75%, relative to the conversion and selectivity of the spent catalyst. In another aspect, the spent catalyst has a reduced or lost ethanol selectivity relative to fresh catalyst, and the regenerated catalyst recovers at least 25%, at least 50% or at least 75% of the lost ethanol selectivity. Similarly, the spent catalyst may have a reduced or lost acetic acid conversion relative to fresh catalyst, and the regenerated catalyst recovers at least 25%, at least 50% or at least 75% of the lost acetic acid conversion.

If steam is employed as the regeneration medium, it may be desired to dry the regenerated hydrogenation catalyst prior to using the regenerated hydrogenation catalyst in the primary hydrogenation process. The drying is optionally performed at a temperature from 10 to 350° C., e.g., 50 to 250° C., from 70 to 180° C. or from 80 to 130° C., and optionally at an absolute pressure from 0.5 to 5 bar, e.g., from 0.8 to 2 bar, or from 0.9 to 1.5 bar, and optionally over a period of time from 10 to 50 hours, e.g., 10 to 20 hours, as described in US Pub. No. 2011/0144398, the entirety of which is incorporated herein by reference.

The following examples describe the catalyst and process of this invention.

### EXAMPLES

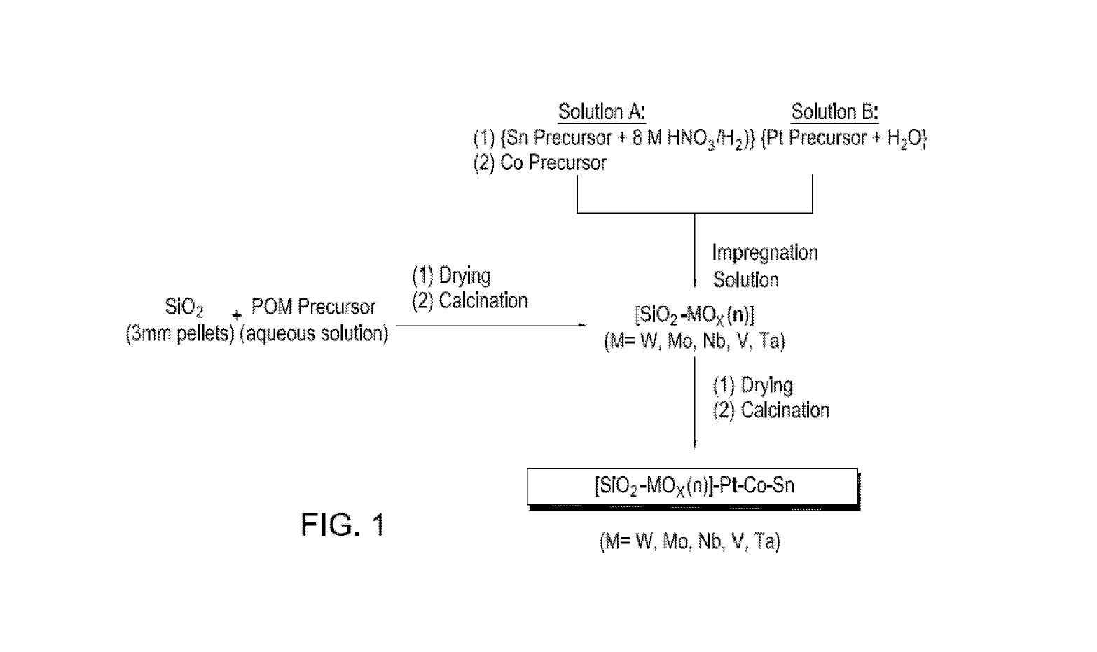

A summary of the catalyst preparation protocol is provided in FIG. 1. Three modified tungsten oxide supported catalysts were prepared with different tungsten oxide loadings as follows.

### Example 1

### SiO₂—WO₃(8)

To obtain 100 g of modified silica support containing 8.0 wt. % WO₃, 8.50 g of Ammonium metatungstate hydrate (AMT) was dissolved in 101 mL of DI-H₂O. The AMT aqueous solution was impregnated onto 92.00 g of silica support. The impregnated material was dried in a rotovaporator for two hours and then placed in a preheated oven at 120° C. for 12 hrs, and calcined in a calcination furnace at 550° C. for 6 hrs.

### Example 2

### SiO₂—WO₃(12)

To obtain 45.45 g of modified silica support containing 12.0 wt. % WO₃, 5.79 g of AMT was dissolved in 45 mL of DI-H₂O. The AMT aqueous solution was impregnated onto 40.00 g of silica support. The impregnated material was dried in a rotovaporator for two hours and then placed in a preheated oven at 120° C. for 12 hrs, and calcined in a calcination furnace at 550° C. for 6 hrs.

### Example 3

### SiO₂—WO₃(16)

To obtain 119.05 g of modified silica support containing 15.3 wt. % WO₃, 19.30 g of AMT was dissolved in 112.50 mL of DI-H₂O. The AMT aqueous solution was impregnated onto 100.00 g of silica support. The impregnated material was dried in a rotovaporator for two hours and then placed material in a preheated oven at 120° C. for 12 hrs, and calcined in a calcination furnace at 550° C. for 6 hrs.

### Examples 4-8

### Catalysts on WO₃-Modified Supports

Catalysts containing tungsten oxide modified supports from Examples 9-11 were prepared as follows.

### Example 4

### Pt(1)Co(4.8)Sn(4.1)/SiO₂—WO₃(8)

Solution A was prepared by adding 9 g of 8M HNO₃ into 4.3225 g of Co(NO₃)₂.6H₂O salt. The solution was further diluted by adding 7 g of DI-H₂O, and 1.3159 g of SnC₂O₄ was added and completely dissolved.

Solution B was prepared by placing 2.0002 g of 10 wt. % Pt oxalate solution in a beaker and adding 6 g of DI-H₂O.

Solution B was added to solution A drop by drop while stirring and the resulting mixed metal solution was stirring for five minutes after addition. The combined solution was added to 16.55 g of SiO₂—WO₃(8) (from Example 1) and dried in a rotovaporator for 1 hr, followed by drying in an oven with preset temperature at 120° C. for 12 hours. The calcination was carried out in a furnace with temperature program from room temperature to 160° C. at 3° C./min and holding at 160° C. for 2 hours, followed by ramping to 350° C. at 3° C./min and holding at 350° C. for 6 hours.

### Example 5

### Pt(1)Co(4.8)Sn(4.1)/SiO₂—WO₃ (12)

Solution A was prepared by adding 9 g of 8M HNO₃ into 1.3157 g of SnC₂O₄ drop by drop. The solution was further diluted by adding 7 g of DI-H₂O. 4.3225 g of Co(NO₃)₂.6H₂O salt was added into the solution slowly while stirring.

Solution B was formed by placing 2.0000 g of 10 wt. % Pt oxalate solution in a beaker and adding 6 g of DI-H₂O.

Solution B was added to solution A drop by drop while stirring. The resulting mixed precursor solution was further stirred for another five minutes. The combined solution was impregnated to 16.55 g of SiO₂—WO₃ (12) (from Example 2), dried in a rotovaporator for 1 hr, and then placed in a drying oven with preset temperature at 120° C. for 12 hours. Calcination was carried out in a furnace with a temperature program from room temperature to 160° C. at 3° C./min and maintained at 160° C. for 2 hours, followed by ramping to 350° C. at 3° C./min and maintained at 350° C. for 6 hours.

### Example 6

### Pt(1)Co(4.8)Sn(4.1)/SiO₂—WO₃ (16)

This catalyst was made in a very similar way as the catalyst of Example 13, except using SiO₂—WO₃ (16) (from Example 3) as support.

### Example 7

### Pt(1)Co(4.8)Sn(4.1)/SiO₂—WO₃ (8)

This catalyst was made in a very similar way as the catalyst of Example 13, except using SiO₂—WO₃ (8) (from Example 1) as support.

### Example 8

### Pt(1.09)Co(4.8)Sn(4.1)/SiO₂—WO₃ (12)

A metal impregnation solution was prepared. A tin salt solution was prepared by dissolving 1.86 g (5.31 mmol) of Sn(IV)Cl₄.5H₂O (solid) into 9.00 g of DI-H₂O. 3.60 g (12.36 mmol) of Co(NO₃)₂.6H₂O solid was added to the solution with stifling. A platinum salt solution was simultaneously prepared by dissolving 0.43 g (0.83 mmol Pt) of H₂PtCl₆.XH₂O (solid, Pt: 38.2 wt. %) into 5.00 g of DI-H₂O. The platinum salt solution was added to the above Co/Sn solution. The mixture was stirred at 400 rpm for 5 minutes at room temperature.

The resulting solution was then added to 13.51 g of WO₃(12)/SiO₂ pellets formed according to Example 2 in a one-liter round flask by using incipient wetness techniques to provide a uniform distribution on the support. After adding the metal solution, the material was evacuated to dryness with a rotary evaporator at a bath temperature of 80° C. and vacuum at 72 mbar for 2 hours, followed by drying at 120° C. at 12 hours under circulating air and calcination at 350° C. for 8 hours. Temperature program: increase from room temperature to 160° C. at 3° C./min ramp, hold at 160° C. for 2 hours, increase from 160° C. to 350° C. at 3° C./min ramp, and hold at 350° C. for 8 hours.

### Example 9

The catalysts of Examples 4-8 was then fed to a test unit as follows. The test unit comprised four independent tubular fixed bed reactor systems with common temperature control, pressure and gas and liquid feeds. The reactors were made of ⅜ inch (0.95 cm) 316 SS tubing, and were 12⅛ inches (30.8 cm) in length. The vaporizers were made of ⅜ inch (0.95 cm) 316 SS tubing and were 12⅜ inches (31.45 cm) in length. The reactors, vaporizers, and their respective effluent transfer lines were electrically heated (heat tape).

The reactor effluents were routed to chilled water condensers and knock-out pots. Condensed liquids were collected automatically, and then manually drained from the knock-out pots as needed. Non-condensed gases were passed through a manual back pressure regulator (BPR) and then scrubbed through water and vented to the fume hood. For each Example, 15 ml of catalyst (3 mm pellets) was loaded into reactor. Both inlet and outlet of the reactor were filled with glass beads (3 mm) to form the fixed bed. The following running conditions for catalyst screening were used: T=275° C., P=300 psig (2068 kPag), [Feed]=0.138 ml/min (pump rate), and [H₂]=513 sccm, gas-hourly space velocity (GHSV)=2246 hr⁻¹. The mixed feed composition used for testing contained 69.92 wt. % acetic acid, 20.72 wt. % ethyl acetate, 5.7 wt. % ethanol, 2.45 wt. % diethyl acetal, 0.65 wt. % water, and 0.55 wt. % acetaldehyde.

The crude product was then analyzed by gas chromatography (Agilent GC Model 6850), equipped with a flame ionization detector. The concentration of acetone was less than 0.1 wt. %. The GC analytical results of the liquid product effluent, excluding water, are provided below in Table 2.

TABLE 2
Liquid Product Effluent Compositions
Examples 4-8 (Pt(1)Co(4.8)Sn(4.1)/Support)

| | | EtOH | EtOAc | AcH | DEE | HOAc | Acetal |
|---|---|---|---|---|---|---|---|
| | | (wt. | (wt. | (wt. | (wt. | (wt. | (wt. |
| Ex. | Support | %) | %) | %) | %) | %) | %) |
| 4 | SiO₂—WO₃ (8) | 60.3 | 16.7 | 0.9 | >0.1 | 0.5 | 0.1 |
| 5 | SiO₂—WO₃ (12) | 61.5 | 15.6 | 0.9 | 0.1 | 0.4 | 0.1 |
| 6 | SiO₂—WO₃ (16) | 63.8 | 12.9 | 0.8 | 0.3 | 0.2 | 0.1 |
| 7 | SiO₂—WO₃ (8) | 58.1 | 17.2 | 0.8 | 0.1 | 1.2 | 0.2 |
| 8 | SiO₂—WO₃ (12) | 63.7 | 12.8 | 0.9 | 0.1 | 0.4 | 0.1 |

Catalyst performance results were then calculated, and are provided below in Table 3.

TABLE 3
Catalyst Performance Data Obtained Under Mixed Feed Conditions
Examples 4-8 (Pt(1)Co(4.8)Sn(4.1)/Support)

| | | HOAc | EtOAc | EtOH | EtOH | EtOH |
|---|---|---|---|---|---|---|
| | | Conv. | Conv. | Select. | Prod. | Prod. |
| Ex. | Support | (%) | (%) | (mol %) | (g/kg/h) | (g/L/h) |
| 4 | SiO₂—WO₃ (8) | 99.3 | 18.4 | 97.1 | 639.4 | 295.3 |
| 5 | SiO₂—WO₃ (12) | 99.4 | 23.7 | 97.1 | 626.2 | 302.7 |
| 6 | SiO₂—WO₃ (16) | 99.7 | 37.0 | 96.1 | 595.5 | 293.9 |
| 7 | SiO₂—WO₃ (8) | 98.2 | 15.7 | 95.0 | 566.9 | 270.0 |
| 8 | SiO₂—WO₃ (12) | 99.5 | 38.1 | 94.5 | 625.8 | 311.2 |

### Short Term Life Analysis

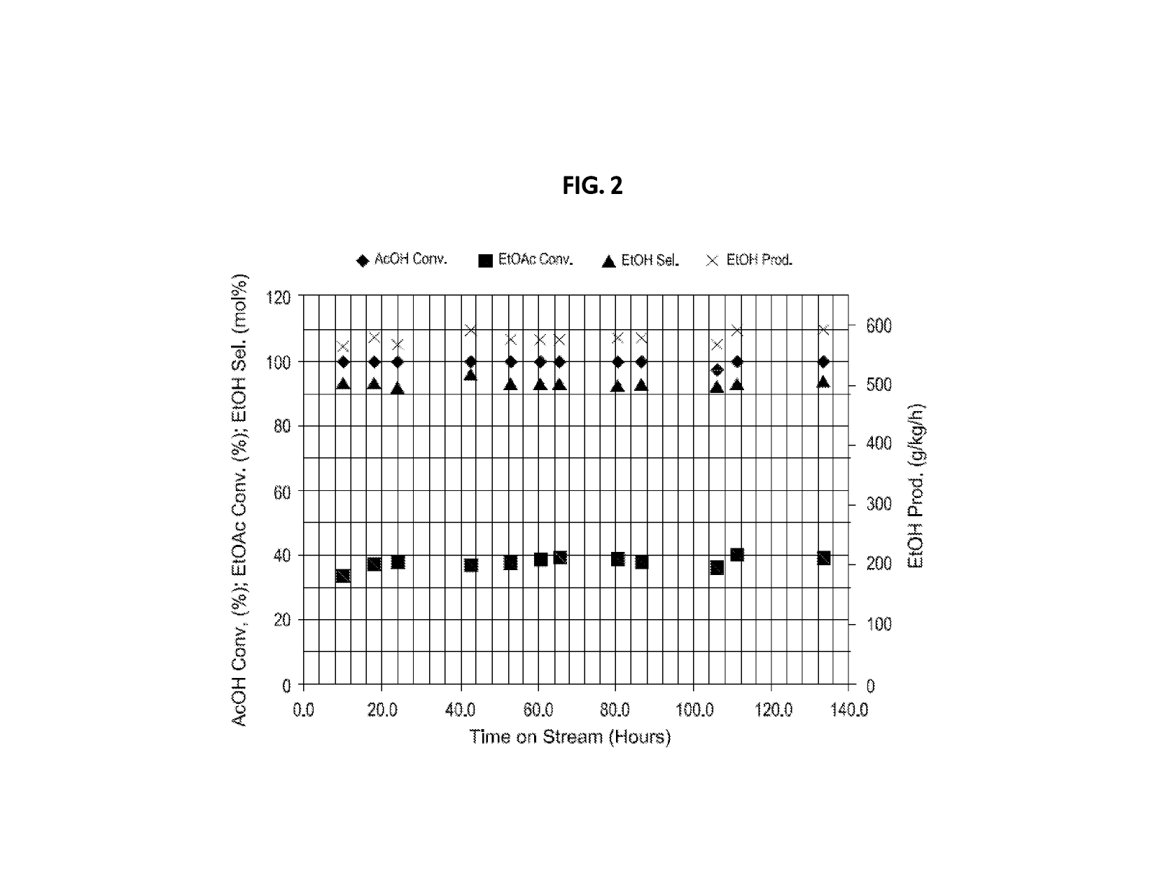

An on-line reduction of the catalyst of Example 5 was implemented with 10% H₂ (N₂ as balance gas) at 275° C. for 30 minutes. Then the catalyst was tested under standard running conditions, as described above. After testing for 43 hours, the unit was shut down under normal shut down conditions. After cooling to room temperature, the unit was restarted and the temperature of reactor was increased to 300° C. An on-line reduction was carried out again under this temperature with 10% H₂ for 3 hours. The results of the two tests were compiled and are indicated in FIG. 2.

The catalyst provided a greater than 99% acetic acid conversion, greater than 90% ethanol selectivity and about 40% ethyl acetate conversion. There was no sign of deactivation of this catalyst after 133 hours test. The series of catalysts with different WO₃ loadings were also tested under standard running conditions but shorter time. They all provided very good activity, selectivity and short term stability.

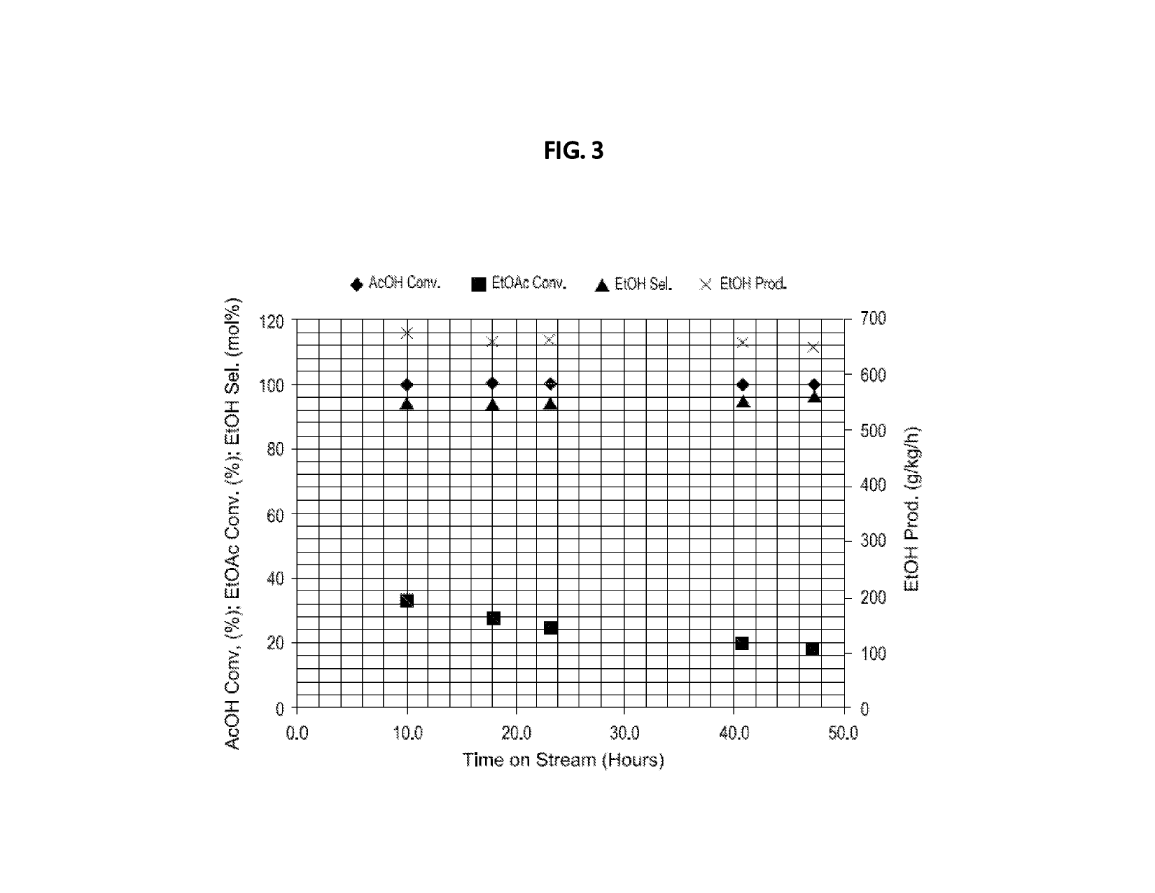

A comparative catalyst comprising Pt(1)Co(4.8)Sn(4.1) on silica, without a modifier, was reduced with 10% H₂ at 275° C. for 30 minutes and tested under standard running conditions, as described above. The results of this test are indicated in FIG. 3. The catalyst provided greater than 99% acetic acid conversion, greater than 90% ethanol selectivity and about 17% ethyl acetate conversion. However, the catalyst showed a noticeable drop in ethyl acetate conversion with running time.

### XRD Characterization

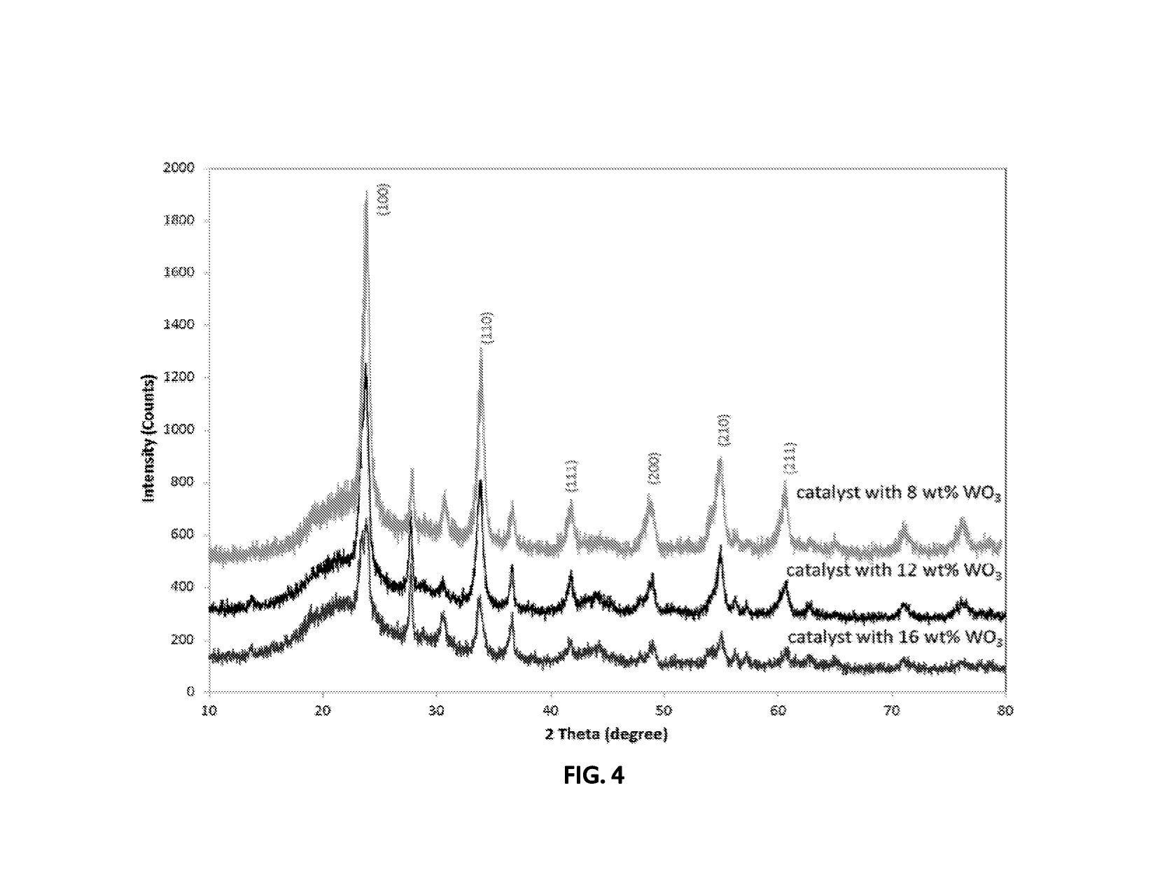

The catalysts from Examples 5-7 were also characterized by X-ray diffraction (XRD). XRD patterns of the samples were obtained using a Rigaku D/Max Ultima II Powder X-ray Diffractometer employing Cu Ka radiation. The X-ray tube was operated at 40 kV and 40 mA. The reduction pretreated catalysts were identified to contain the cubic tungsten oxide (H_{0.5}WO₃; Entry #: 28691-ICSD) as the major phase as shown in FIG. 4.

An x-ray diffraction pattern substantially as shown in Table 4:

TABLE 4

| | | Relative |
|---|---|---|
| 2θ (°, ± 0.30) | d-spacing (Å) | Intensity |
| 24.07 | 3.69 | 100.00 |
| 27.97 | 3.19 | 22.50 |
| 34.04 | 2.63 | 62.00 |
| 36.80 | 2.44 | 12.80 |
| 42.02 | 2.15 | 18.00 |
| 48.91 | 1.86 | 13.50 |
| 55.18 | 1.66 | 25.90 |
| 60.75 | 1.52 | 17.90 |
| 71.36 | 1.32 | 7.00 |
| 76.65 | 1.24 | 9.30 |

A catalyst comprising cobalt, a precious metal and at least one active metal on a modified support comprising tungsten oxide, wherein said catalyst has an x-ray diffraction pattern in which above 2θ=10°, there is a local maximum having a characteristic full width at a half maximum at each of: a 2θ value in the range from 23.54 to 24.60°; a 2θ value in the range from 27.81 to 28.13°; a 2θ value in the range from 33.52 to 34.56°; a 2θ value in the range from 41.62 to 42.42°; a 2θ value in the range from 54.70 to 55.66°; a 2θ value in the range from 60.18 to 61.32°.

While the invention has been described in detail, modifications within the spirit and scope of the invention will be readily apparent to those skilled in the art. All publications and references discussed above are incorporated herein by reference. In addition, it should be understood that aspects of the invention and portions of various embodiments and various features recited may be combined or interchanged either in whole or in part. In the foregoing descriptions of the various embodiments, those embodiments which refer to another embodiment may be appropriately combined with other embodiments as will be appreciated by one skilled in the art. Furthermore, those skilled in the art will appreciate that the foregoing description is by way of example only, and is not intended to limit the invention.

1. A catalyst, comprising:
cobalt, a precious metal and at least one active metal on a modified support,
wherein the precious metal is selected from the group consisting of rhodium, rhenium, ruthenium, platinum, palladium, osmium, iridium and gold;
wherein the at least one active metal is selected from the group consisting of copper, iron, nickel, titanium, zinc, chromium, tin, lanthanum, cerium, and manganese; and
wherein the modified support comprises (i) support material; and (ii) a support modifier comprising a metal selected from the group consisting of tungsten, molybdenum, vanadium, niobium, and tantalum.

2. The catalyst of claim 1, wherein the precious metal is present in an amount from 0.1 to 5 wt. %, cobalt is present in an amount from 0.5 to 20 wt. % and the at least one active metal is present in an amount from 0.5 to 20 wt. %, based on the total weight of the catalyst.

3. The catalyst of claim 1, wherein the catalyst comprises an oxide of tungsten, molybdenum or vanadium in an amount from 0.1 to 40 wt. %.

4. The catalyst of claim 1, wherein the support modifier comprises tungsten oxide.

5. The catalyst of claim 1, wherein the support modifier is substantially free of cobalt and/or the at least one active metal.

6. The catalyst of claim 1, wherein the at least one active metal is selected from the group consisting of copper, iron, nickel, zinc, chromium, and tin.

7. The catalyst of claim 1, wherein the precious metal is palladium and/or platinum, and the at least one active metal is tin.

8. The catalyst of claim 1, wherein the support material is selected from the group consisting of silica, alumina, titania, silica/alumina, pyrogenic silica, high purity silica, zirconia, carbon, zeolites and mixtures thereof.

9. A process for producing ethanol, comprising contacting a feed stream comprising acetic acid and hydrogen in a reactor at an elevated temperature in the presence of the catalyst of claim 1, under conditions effective to form ethanol.

10. The process of claim 9, wherein the feed stream further comprises ethyl acetate in an amount greater than 5 wt. %.

11. The process of claim 9, wherein the feed stream further comprises ethyl acetate in an amount greater than 5 wt. %, wherein acetic acid conversion is greater than 20% and ethyl acetate conversion is greater than 5%.

12. The process of claim 9, wherein acetic acid conversion is at least 80%.

13. The process of claim 9, wherein acetic acid selectivity to ethanol is greater than 80%.

14. The process of claim 9, wherein the process forms a crude product comprising the ethanol and ethyl acetate, and wherein the crude product has an ethyl acetate steady state concentration from 0.1 to 40 wt. %.

15. The process of claim 9, wherein the acetic acid is formed from methanol and carbon monoxide, wherein each of the methanol, the carbon monoxide, and hydrogen for the hydrogenating step is derived from syngas, and wherein the syngas is derived from a carbon source selected from the group consisting of natural gas, oil, petroleum, coal, biomass, and combinations thereof.

16. A synthesis process for producing the catalyst of claim 1,
(a) impregnating a support material with a support modifier precursor to form a first impregnated support, wherein the support modifier precursor comprises a support modifier metal selected from the group consisting of tungsten, molybdenum, niobium, vanadium and tantalum;
(b) heating the first impregnated support to a first temperature to form a modified support;
(c) impregnating the modified support with a second mixed precursor to form a second impregnated support, wherein the second mixed precursor comprises precursors to cobalt, the precious metal, and the at least one active metal; and
(d) heating the second impregnated support to a second temperature to form the catalyst.

17. The synthesis process of claim 16, wherein the second temperature is less than the first temperature.

18. The synthesis process of claim 16, wherein the second temperature is at least 50° C. less than the first temperature.

19. The synthesis process of claim 16, wherein the second temperature is at least 100° C. less than the first temperature.

20. A catalyst, comprising:
a modified support comprising a silicaceous support material and a support modifier comprising a support modifier metal selected from the group consisting of tungsten, molybdenum, niobium, vanadium and tantalum, and
a first metal, a second metal and a third metal on the modified support, wherein the first metal is a precious metal selected from the group consisting of rhodium, rhenium, ruthenium, platinum, palladium, osmium, iridium and gold, and at least one of the second or third metal is cobalt, and wherein the first metal is present in an amount from 0.1 to 5 wt. %, the second metal is present in an amount from 0.5 to 20 wt. % and the third metal is present in an amount from 0.5 to 20 wt. %, based on the total weight of the catalyst.

21. The catalyst of claim 20, wherein the second or third metals are selected from the group consisting of cobalt, copper, iron, nickel, titanium, zinc, chromium, tin, lanthanum, cerium, and manganese.

22. A hydrogenation catalyst comprising cobalt, a precious metal and at least one active metal on a modified support comprising tungsten oxide, and having, after calcination, an x-ray diffraction pattern substantially as shown in the following Table:

| | | Relative |
|---|---|---|
| 2θ (°, ± 0.30) | d-spacing (Å) | Intensity |
| 24.07 | 3.69 | 100.00 |
| 27.97 | 3.19 | 22.50 |
| 34.04 | 2.63 | 62.00 |
| 36.80 | 2.44 | 12.80 |
| 42.02 | 2.15 | 18.00 |
| 48.91 | 1.86 | 13.50 |
| 55.18 | 1.66 | 25.90 |
| 60.75 | 1.52 | 17.90 |
| 71.36 | 1.32 | 7.00 |
| 76.65 | 1.24 | 9.30 |

23. A catalyst comprising cobalt, a precious metal and at least one active metal on a modified support comprising tungsten oxide, and having, after calcination, an x-ray diffraction pattern in which above 2θ=10°, there is a local maximum having a characteristic full width at a half maximum at each of: a 2θ value in the range from 23.54 to 24.60°; a 2θ value in the range from 27.81 to 28.13°; a 2θvalue in the range from 33.52 to 34.56°; a 2θ value in the range from 41.62 to 42.42°; a 2θ value in the range from 54.70 to 55.66°; a 2θ value in the range from 60.18 to 61.32°.
